(19) United States
(12) Patent Application Publication
(10) Pub. No.: US 2017/0071463 A1
(43) Pub. Date: Mar. 16, 2017

(54) TONGUE HOLDER

(71) Applicant: HTC Corporation, Taoyuan City (TW)

(72) Inventors: Bo-Wen XIAO, Taoyuan City (TW); Ming-Tien LIN, Taoyuan City (TW); Chih-Jen HU, Taoyuan City (TW); Yi-Chi LIN, Taoyuan City (TW); Tun-Chun YANG, Taoyuan City (TW)

(73) Assignee: HTC Corporation, Taoyuan City (TW)

(21) Appl. No.: 14/852,175

(22) Filed: Sep. 11, 2015

(51) Int. Cl. A61B 1/24; H04N 5/225; A61B 1/06; A61B 5/00; A61B 1/04; H04N 5/232; A61B 13/00

(52) U.S. Cl. CPC A61B 1/24; H04N 5/232; H04N 5/2256; A61B 13/00; A61B 5/0088; A61B 1/04; A61B 1/06; H04N 2005/2255

(57) ABSTRACT

A tongue holding device is provided. The tongue holding device is for pressing a tongue within a mouth cavity to capture an image of the mouth cavity. The tongue holding device includes a holder, a tongue pressing portion and an image capturing element. The tongue pressing portion and the image capturing element are disposed on the holder.

### TECHNICAL FIELD

The disclosure relates in general to a tongue holding device, and more particularly to a tongue holding device having an image capturing element.

### BACKGROUND

Conventionally, when a doctor checks the tissues of a patient's mouth cavity, the doctor will press the patient's tongue with a stick held at one hand and check the patient's mouth cavity with a flashlight held at the other hand. However, such operation is very inconvenient. Therefore, it has become an urgent need to provide a solution for conveniently checking the tissues of the mouth cavity.

### SUMMARY

The disclosure is directed to a tongue holding device capable of increasing the convenience for checking the tissues of the mouth cavity.

According to one embodiment, a tongue holding device is provided. The tongue holding device is for pressing a tongue within a mouth cavity to capture an image of the mouth cavity. The tongue holding device includes a holder, a tongue pressing portion and an image capturing element. The tongue pressing portion is disposed on the holder. The image capturing element is protrudingly disposed on the holder.

The above and other aspects of the disclosure will become better understood with regard to the following detailed description of the preferred but non-limiting embodiment(s). The following description is made with reference to the accompanying drawings.

### BRIEF DESCRIPTION OF THE DRAWINGS

FIG. 1A is a side view of a tongue holding device according to an embodiment of the present disclosure.

FIG. 1B is a top view of the tongue holding device of FIG. 1A.

FIG. 2A is a side view of a tongue holding device according to another embodiment of the present disclosure.

FIG. 2B is a partial appearance view of the tongue holding device of FIG. 2A.

FIG. 3 is a side view of a tongue holding device according to another embodiment of the present disclosure.

FIG. 4 is a side view of a tongue holding device according to another embodiment of the present disclosure.

FIG. 5A is a top view of a tongue holding device according to another embodiment of the present disclosure.

FIG. 5B is a side view of the tongue holding device of FIG. 5A.

FIG. 6 is an appearance view of a tongue holding device according to another embodiment of the present disclosure.

FIG. 7 is an appearance view of a tongue holding device of according to another embodiment of the present disclosure.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

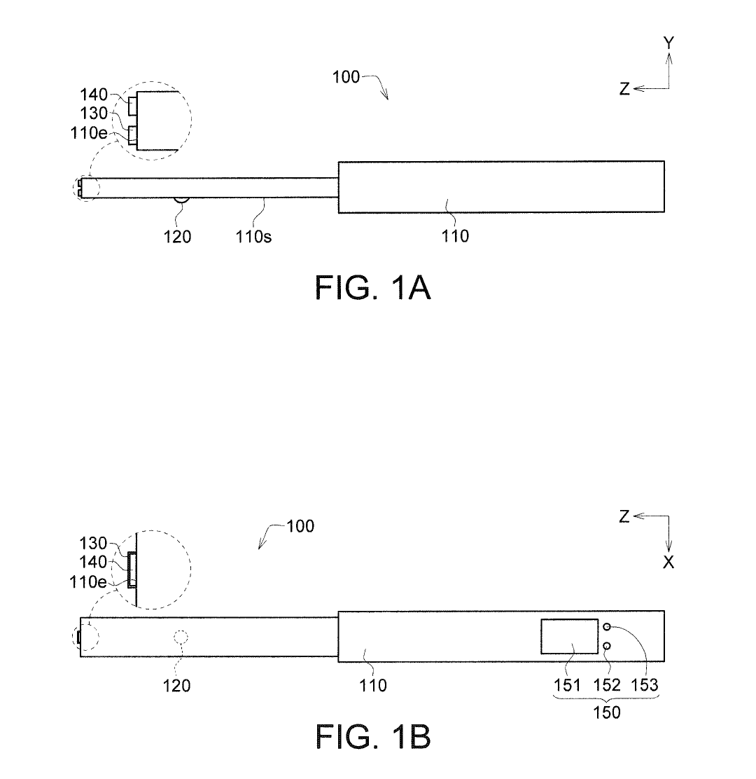

FIG. 1A is a side view of a tongue holding device 100 according to an embodiment of the present disclosure. FIG. 1B is a top view of the tongue holding device 100 of FIG. 1A. The tongue holding device 100 is for pressing a tongue within a mouth cavity to capture an image of the mouth cavity.

The tongue holding device 100 includes a holder 110, a tongue pressing portion 120, an image capturing element 130, an illuminator 140, and a control circuit 150. Since both the tongue pressing portion 120 and the image capturing element 130 of the present embodiment are integrated in the same holder 110, the tongue holding device 100 can concurrently achieve the effects of pressing the tongue to expose the mouth cavity and capturing an image of the mouth cavity. Since the tongue pressing portion 120 and the image capturing element 130 are integrated in the same holder 110, the user can achieve the above two effects by operating the tongue holding device 100 with one single hand. Detailed structure of the tongue holding device 100 is disclosed below.

As indicated in FIGS. 1A and 1B, the holder 110 is flat. In the present embodiment, the cross-section of the holder 110 can be a rectangle, a circle, an ellipse or a polygon.

As indicated in FIG. 1A, the holder 110 has a peripheral surface 110s on which the tongue pressing portion 120 is disposed. In the present embodiment, the tongue pressing portion 120 is a bump. As indicated in FIG. 1B, the entire tongue pressing portion 120 overlaps the holder 110, such that the tongue holding device 100 has a smaller volume or length, hence possessing better portability and receivability.

The image capturing element 130 can capture an image of the tissues of a mouth cavity, such as the throat tissues. Thus, the user (such as a doctor) can check the tissues of the mouth cavity according to the captured image without directly checking the tissues of the mouth cavity using his/her naked eyes. In an embodiment, the image capturing element 130 can be realized by a camera or a video recorder. Besides, the image capturing element 130 is disposed on an end surface 110e of the holder 110 such that the illuminator 140 can capture the image in a direction pointed by the end surface 110e of the holder 110. Furthermore, the tongue holding device 100 can capture the image of the mouth cavity right in front of the end surface 110e of the holder 110. The user can capture a desired image of the mouth cavity by pointing the end surface 110e of the holder 110 towards the to-be-checked tissues. For example, if the user wants to check the patient's throat, the user only needs to point the end surface 110e of the holder 110 towards the throat to capture the image of the throat tissues. Since the tongue pressing portion 120 is disposed on the peripheral surface 110s of the holder 110, the user can use the tongue pressing portion 120 to push away the tongue to avoid the shooting sight of the image capturing element 130 being blocked by the tongue.

The illuminator 140 can illuminate the mouth cavity for the user to clearly check the tissues of the mouth cavity. The illuminator 140 is disposed on the end surface 110e of the holder 110, such that the illuminator 140 can emit a light in the direction pointed by the end surface 110e of the holder 110. Furthermore, the illuminator 140 can be used as a fill-in light for improving the quality of the image captured by the image capturing element 130. Moreover, the illuminator 140 can be disposed above, beneath, to the left, or to the right of the image capturing element 130. In the present embodiment, the quantity of the illuminator 140 can be one or more than one. If the quantity of the illuminator 140 is more than one, the illuminators 140 can be disposed surrounding the image capturing element 130.

The control circuit 150 can control the image capturing element 130 to start or stop capturing image and/or control the illuminator 140 to start or stop emitting a light. In the present embodiment, the control circuit 150 includes a monitor 151, a first touch key 152 and a second touch key 153.

The monitor 151 can display the picture or dynamic image captured by the image capturing element 130. Or, the monitor 151 can display a number of indicator lights respectively representing the connection status between the tongue holding device 100 and a number of external devices such as a notebook computer, a desktop computer, a mobile phone and a display. For example, when the tongue holding device 100 and the display are connected, a corresponding indicator light will be turned on to indicate that the tongue holding device 100 and the display are connected. The display connected to the tongue holding device 100 can display the picture or dynamic image captured by the image capturing element 130.

The first touch key 152 is for operating the image capturing element 130, and can be realized by such as a shutter. The second touch key 153 is for operating the illuminator 140, and can be realized by such as a light source power key. In addition, the first touch key 152 can provide multiple functions. For example, the first touch key 152 can provide different functions according to the duration for which the first touch key 152 is continuously activated. In another embodiment, the first touch key 152 can be used as a shutter and a power key. Under such design, the first touch key 152 can turn on/off the power of the tongue holding device 100 if the first touch key 152 is pressed for a longer duration, and can start or stop capturing an image if the first touch key 152 is pressed for a shorter duration. Similarly, the second touch key 153 also can provide multiple functions, and the control methods are similar to that of the first touch key 152, and the similarities are not repeated here.

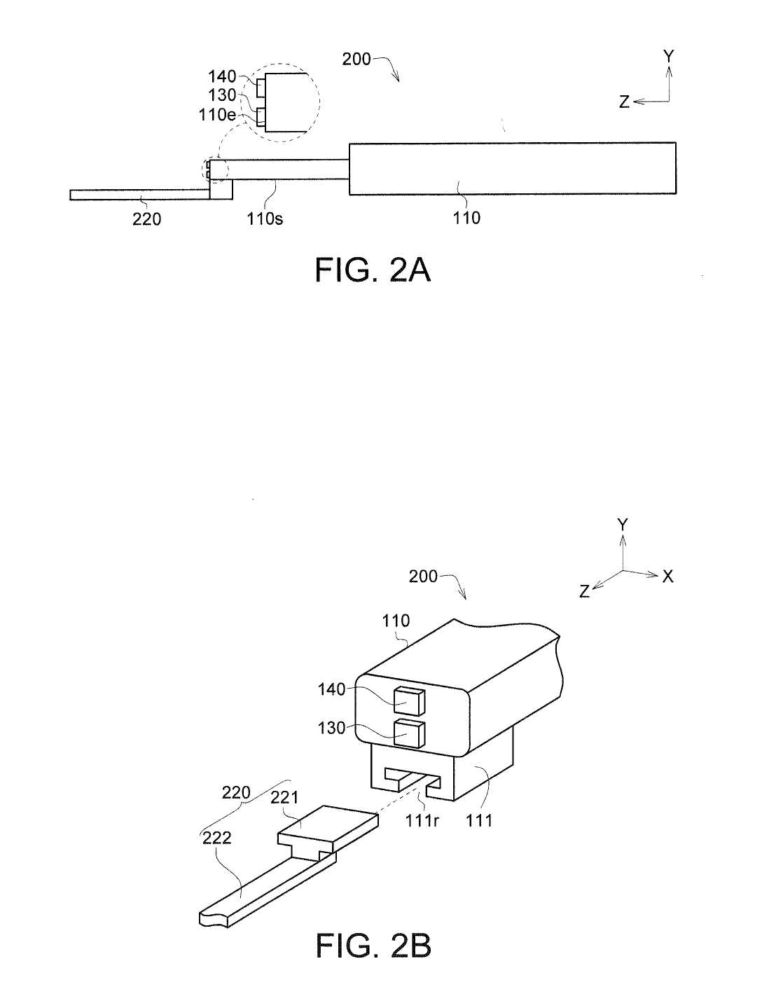

FIG. 2A is a side view of a tongue holding device 200 according to another embodiment of the present disclosure. FIG. 2B is a partial appearance view of the tongue holding device 200 of FIG. 2A. The tongue holding device 200 includes a holder 110, a tongue pressing portion 220, an image capturing element 130, an illuminator 140, and a control circuit 150.

The tongue pressing portion 220 includes a connecting portion 221 and a pressing portion 222 connected to each other. The tongue pressing portion 220 is connected to the holder 110 through the connecting portion 221. The connecting portion 221 is connected to but not parallel to the pressing portion 222. As indicated in FIG. 2A, the connecting portion 221 and the pressing portion 222 of the present embodiment form an L shape. In another embodiment, the connecting portion 221 and the pressing portion 222 can form a V shape or other non-parallel shape. Besides, the connecting portion 221 of the tongue pressing portion 220 may include a number of non-parallel sub-connecting portions, such that the tongue pressing portion 220 can form an even more complicated shape such as an M shape or a U shape.

As indicated in FIG. 2A, the tongue pressing portion 220 exceeds the end surface 110e of the holder 110 to increase the length of the tongue holding device 200 such that the tongue pressing portion 220 can reach deeper inside the mouth cavity.

As indicated in FIG. 2B, the tongue pressing portion 220 is separably disposed on the holder 110. Specifically, the tongue pressing portion 220 has one of a slide track and a slide chute, the holder 110 has the other one of the slide track and the slide chute, and the tongue pressing portion 220 and the holder 110 are fixed to each other through the slide track and the slide chute being engaged with each other. For example, the connecting portion 221 of the tongue pressing portion 220 is a slide track, the connecting portion 111 of the holder 110 is a slide chute 111r, and the tongue pressing portion 220 and the holder 110 are fixed to each other through the connecting portion 221 and the slide chute 111r being engaged with each other.

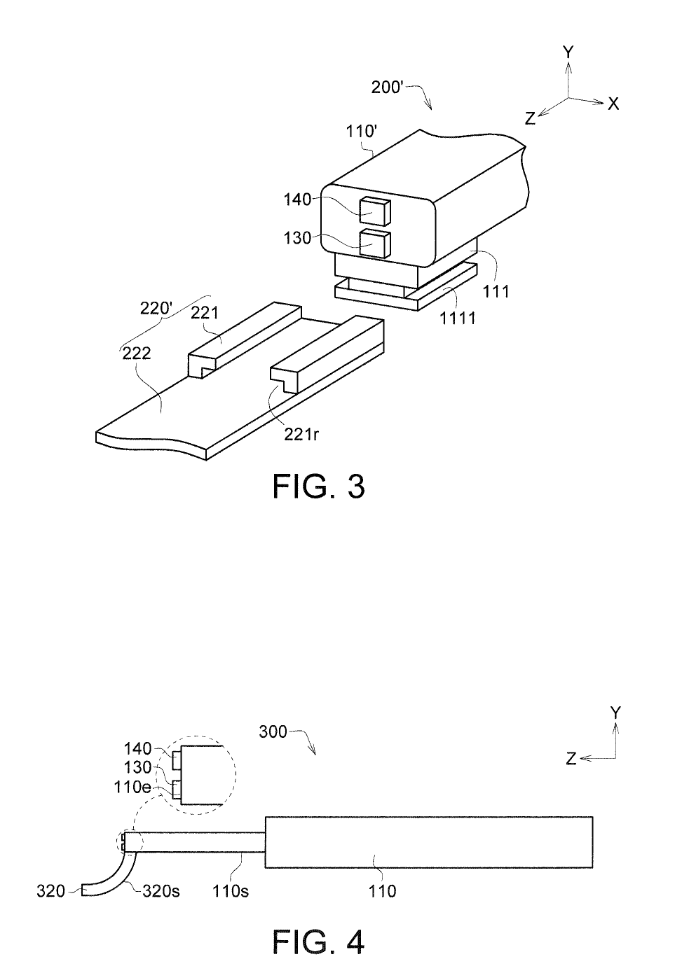

FIG. 3 is a side view of a tongue holding device 200′ according to another embodiment of the present disclosure. The tongue holding device 200′ includes a holder 110′, a tongue pressing portion 220′, an image capturing element 130, an illuminator 140, and a control circuit 150 (not illustrated). In the present embodiment, the connecting portion 111 of the holder 110′ has a slide track 1111, the connecting portion 221 of the tongue pressing portion 220′ has a slide chute 221r, and the tongue pressing portion 220′ and the holder 110′ are fixed to each other through the slide chute 221r and the slide track 1111 being engaged with each other.

FIG. 4 is a side view of a tongue holding device 300 according to another embodiment of the present disclosure. The tongue holding device 300 includes a holder 110, a tongue pressing portion 320, an image capturing element 130, an illuminator 140, and a control circuit 150. In the present embodiment, the tongue pressing portion 320 presses the tongue by a curved surface 320s. The curved surface 320s can increase the contact area with the tongue, so that the user can effortlessly press the tongue. Moreover, the curved surface 320s can match the shape of the tongue, such that the contact area between the curved surface 320s and the tongue can be maximized, and the user can press the tongue even more effortlessly.

The tongue pressing portion 320 can be a flexible tongue pressing portion or a hard tongue pressing portion. Let the flexible tongue pressing portion be taken for example. The flexible tongue pressing portion can match the deformation the tongue when pressed, such that the contact area between the flexible tongue pressing portion the tongue can be increased. In an embodiment, the tongue pressing portion 320 can be formed of rubber, plastics or metal. However, the material of the tongue pressing portion 320 is not limited thereto, and the tongue pressing portion 320 can be formed of any materials compactible with or not harmful to human body.

As indicated in FIG. 4, the tongue pressing portion 320 protrudes from the peripheral surface 110s of the holder 110 and exceeds the end surface 110e of the holder 110 to increase the length of the tongue holding device 300, such that the tongue pressing portion 320 can reach deeper inside the mouth cavity.

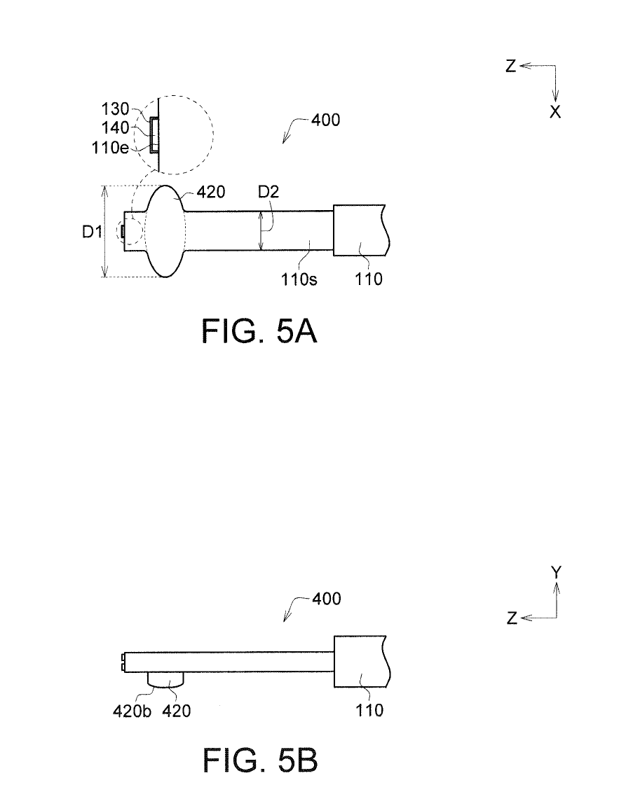

FIG. 5A is a top view of a tongue holding device 400 according to another embodiment of the present disclosure. FIG. 5B is a side view of the tongue holding device 400 of FIG. 5A.

The tongue holding device 400 includes a holder 110, a tongue pressing portion 420, an image capturing element 130, an illuminator 140, and a control circuit 150 (not illustrated). In the present embodiment, the tongue pressing portion 420 has a size D1 along the X axis, the holder 110 has a size D along the same X axis2, and the size D1 is larger than the size D2. That is, a portion of the tongue pressing portion 420 overlaps the holder 110, but the other portion of the tongue pressing portion 420 does not overlap the holder 110. Since the tongue pressing portion 420 provides a large pressing area, the contact area between the tongue pressing portion 420 and the tongue can be increased. As indicated in FIG. 5A, the shape of the tongue pressing portion 420 can be an ellipse. However, the shape of the tongue pressing portion 420 can be a circle, a rectangle or other shapes.

As indicated in FIG. 5B, the tongue pressing portion 420 is protrudingly disposed on the peripheral surface 110s of the holder 110. In the present embodiment, the tongue pressing portion 420 has a surface 420b, such as a convex surface or a concave surface, by which the tongue pressing portion 420 presses the tongue.

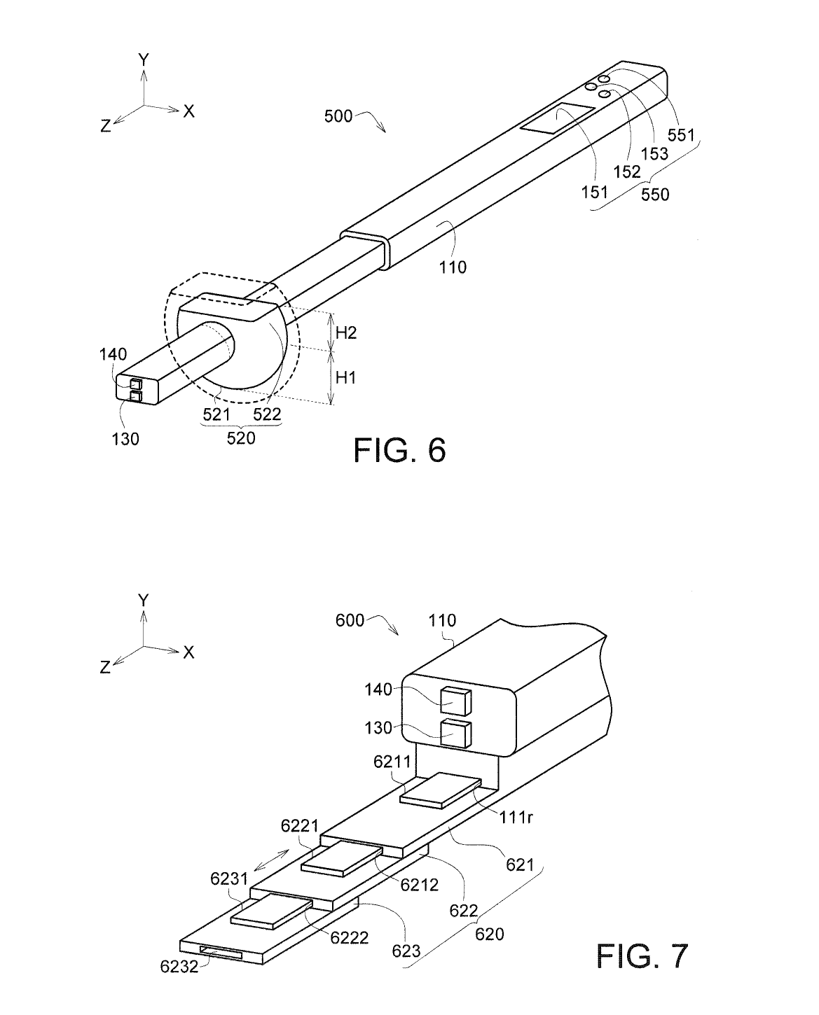

FIG. 6 is an appearance view of a tongue holding device 500 according to another embodiment of the present disclosure. The tongue holding device 500 includes a holder 110, a tongue pressing portion 520, an image capturing element 130, an illuminator 140, and a control circuit 550.

The tongue pressing portion 520 is an expandable portion, such as an airbag. The control circuit 550 further includes a third touch key 551 for controlling the tongue pressing portion 520 to expand or contract. In another embodiment, the control circuit 550 can dispense with the third touch key 551. Under such design, the tongue pressing portion 520 is controlled to expand or contract according to the duration for which one of the first touch key 152 and the second touch key 153 is activated. For example, by pressing one of the first touch key 152 and the second touch key 153 for a longer duration of the time, the tongue pressing portion 520 can be controlled to expand (the expanded tongue pressing portion 520 is illustrated in dotted lines) or contract. Furthermore, one surface of the tongue pressing portion 520 can be a convex surface or a concave surface. The shape of the tongue pressing portion 520 can be a circle, an ellipse, or a polygon such as a rectangle.

Additionally, the tongue pressing portion 520 includes a first portion 521 and a second portion 522, which are respectively disposed on two opposite sides of the holder 110. For example, the first portion 521 is disposed under the holder 110 for pressing the tongue, and the second portion 522 is disposed above the holder 110. The height H2 of the second portion 522 is smaller than the height H1 of the first portion 521. Since the height H2 of the second portion 522 is small enough, the palate of the testee's mouth cavity will not be touched by the second portion 522 and will avoid causing discomfort to the testee.

FIG. 7 is an appearance view of a tongue holding device 600 of according to another embodiment of the present disclosure. The tongue holding device 500 includes a holder 110, a tongue pressing portion 620, an image capturing element 130, an illuminator 140, and a control circuit 150 (not illustrated).

The tongue pressing portion 620 is a scalable member. For example, the tongue pressing portion 620 includes a first rod 621, a second rod 622, and a third rod 623. The first rod 621 has a first slide track 6211 and a first slide chute 6212; the second rod 622 has a second slide track 6221 and a second a slide chute 6222; the third rod 623 has a third slide track 6231 and a third slide chute 6232. The first slide track 6211 of the first rod 621 can be slidably connected to the slide chute 111r of the holder 110; the second slide track 6221 of the second rod 622 can be slidably connected to the first slide chute 6212 of the first rod 621; the third slide track 6231 of the third rod 623 can be slidably connected to the second slide chute 6222 of the second rod 622. Thus, the tongue pressing portion 620 is scalable with respect to the holder 110. In another embodiment, the tongue pressing portion 620 may further include another rod, which can be slidably connected to the third slide track 6231 of the third rod 623.

To summarize, the tongue holding device disclosed in the embodiments of the present disclosure, including a tongue pressing portion and an image capturing element, is capable of achieving both effects of pressing the tongue to expose the mouth cavity and capturing an image of the mouth cavity. In an embodiment, since the tongue pressing portion and the image capturing element are integrated in the same holder, the user can above two effects by operate the tongue holding device with one single hand. In another embodiment, the tongue pressing portion is protrudingly disposed on the peripheral surface of the holder, and will not interfere with the shooting sight of the image capturing element. In another embodiment, it does not matter whether the tongue pressing portion exceeds the end surface of the holder or not. In another embodiment, the tongue pressing portion, possessing flexibility, flexibly matches the hardness or deformation of the tongue. In another embodiment, the tongue pressing portion, possessing scalability, is capable of changing the length according to the depth of the to-be-checked tissues. Or, the tongue pressing portion can be a bump. In the embodiments of the present disclosure, the shape and/or material of the tongue pressing portion are not restricted, and any shapes and/or materials enabling the tongue pressing portion to press the tongue would do. Besides, any one surface of the tongue pressing portion can be a convex surface or a concave surface. In other embodiments, the cross-section of the holder of the tongue holding device can be in the shape of a circle, an ellipse, or a polygon such as a rectangle. In another embodiment, the quantity of the illuminator of tongue holding device can be one or more than one.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A tongue holding device for pressing a tongue within a mouth cavity to capture an image of the mouth cavity, wherein the tongue holding device comprises:
a holder;
a tongue pressing portion disposed on the holder; and
an image capturing element disposed on the holder;
wherein, the holder has a peripheral surface on which the tongue pressing portion is protrudingly disposed.

2. The tongue holding device according to claim 1, wherein the tongue pressing portion comprises a connecting portion and a pressing portion connected to each other, and the connecting portion is connected to the holder but is non-parallel to the pressing portion.

3. The tongue holding device according to claim 2, wherein the connecting portion and the pressing portion form an L shape.

4. The tongue holding device according to claim 1, wherein the tongue pressing portion has a curved surface by which the tongue pressing portion presses the tongue.

5. The tongue holding device according to claim 1, wherein the tongue pressing portion is a bump.

6. The tongue holding device according to claim 1, wherein the tongue pressing portion is a flexible tongue pressing portion.

7. The tongue holding device according to claim 1, wherein the holder has an end surface on which the image capturing element is disposed.

8. The tongue holding device according to claim 1, wherein the holder has an end surface and the tongue pressing portion exceeds the end surface.

9. The tongue holding device according to claim 1, wherein the tongue pressing portion is separably connected to the holder.

10. The tongue holding device according to claim 9, wherein the tongue pressing portion has one of a slide track and a slide chute, the holder has the other one of the slide track and the slide chute, and the tongue pressing portion and the holder are fixed to each other through the slide track and the slide chute being engaged with each other.

11. The tongue holding device according to claim 1, wherein the entire tongue pressing portion overlaps the holder.

12. The tongue holding device according to claim 1, wherein a portion of the tongue pressing portion overlaps the holder, and the other portion of the tongue pressing portion does not overlap the holder.

13. The tongue holding device according to claim 1, wherein the tongue pressing portion is an expandable portion.

14. The tongue holding device according to claim 13, wherein the expandable portion is an airbag.

15. The tongue holding device according to claim 13, further comprising:
a control circuit for controlling the expandable portion to expand or contract.

16. The tongue holding device according to claim 1, further comprising:
a control circuit for controlling the image capturing element to start or stop capturing the image.

17. The tongue holding device according to claim 1, further comprising:
an illuminator for illuminating the mouth cavity.

18. The tongue holding device according to claim 17, further comprising:
a control circuit for controlling the illuminator to start or stop emitting a light.

19. The tongue holding device according to claim 1, wherein the tongue pressing portion is a scalable member.

20. The tongue holding device according to claim 1, wherein the tongue pressing portion has a convex surface or a concave surface.

21. The tongue holding device according to claim 1, wherein the tongue pressing portion comprises a first portion and a second portion respectively disposed on two opposite sides of the holder, and the height of the first portion is different from that of the second portion.
